(19) United States
(12) Patent Application Publication
(10) Pub. No.: US 2024/0319179 A1
(43) Pub. Date: Sep. 26, 2024

(54) MULTIPLEXED NANO-BIOSENSOR SYSTEM FOR EARLY DETECTION OF DIABETES

(71) Applicants: Ghassem Amoabediny, Tehran (IR); Mohammadreza Farrokhnia, Shiraz (IR); Mohammad Arjmand, Kelowna (CA); Mohammad Ebrahimi, Tehran (IR)

(72) Inventors: Ghassem Amoabediny, Tehran (IR); Mohammadreza Farrokhnia, Shiraz (IR); Mohammad Arjmand, Kelowna (CA); Mohammad Ebrahimi, Tehran (IR)

(73) Assignees: Ghassem Amoabediny, Tehran, OT (IR); Mohammadreza Farrokhnia, Shiraz, OT (IR); Mohammad Arjmand, Kelowna, BC (CA); Mohammad Ebrahimi, Tehran, OT (IR); University of Tehran, Tehran, OT (IR)

(21) Appl. No.: 18/393,445

(22) Filed: Dec. 21, 2023

Related U.S. Application Data
(63) Continuation-in-part of application No. PCT/IB2022/054517, filed on May 16, 2022.
(60) Provisional application No. 63/212,730, filed on Jun. 21, 2021.

(51) Int. Cl. G01N 33/543; B01L 3/00

(52) U.S. Cl. CPC G01N 33/5438; B01L 3/50273; B01L 2300/0645; B01L 2300/0809; B01L 2300/0861; B01L 2300/16; B01L 2400/0487; G01N 2333/62; G01N 2333/705; G01N 2333/765

(57) ABSTRACT

A nano-biosensor system for early detection of diabetes includes a plurality of electrochemical immunosensors and a multiplexed microfluidic system that may be connected in fluid communication with the plurality of electrochemical immunosensors. A working electrode of each electrochemical immunosensor includes a base gold surface, a porous layer of polyaniline conductive polymer deposited onto the base gold surface, gold nanoparticles deposited inside pores of the porous layer of polyaniline conductive polymer, L-glutathione reduced linkers attached from respective thiol group ends of the L-glutathione reduced linkers to the gold nanoparticles, and a specific antigen connected to carboxyl group ends of the L-glutathione reduced linkers. The specific antigen includes at least one of recombinant insulin protein, insulin receptor protein, and recombinant glial fibrillary acidic protein.

### CROSS-REFERENCE TO RELATED APPLICATION

This application is a continuation-in-part of PCT/IB2022/054517, filed on May 16, 2022, and entitled “MULTIPLEXED NANO-BIOSENSOR SYSTEM FOR EARLY DETECTION OF DIABETES” which claims the benefit of priority from pending U.S. Provisional Patent Application Ser. No. 63/212,730, filed on Jun. 21, 2021, and entitled “MULTIPLEXED NANO-BIOSENSOR SYSTEM FOR EARLY DETECTION OF DIABETES,” which are both incorporated herein by reference in their entirety.

### TECHNICAL FIELD

The present disclosure generally relates to multiplexed microfluidic devices, particularly to biosensor systems and methods for early detection of diabetes. More particularly, the present disclosure relates to multiplexed nano-biosystems for determining concentration of biofluid antibodies based on electrochemical analysis.

### BACKGROUND

Diabetes Mellitus is a chronic non-communicable metabolic disease that is spreading rapidly around the world. Insulin-deficient diabetes may be caused by the destruction of pancreatic beta cells (type 1 diabetes, hypoglycemia) or a disorder of the body cells' insulin receptors and insulin resistance (type 2 diabetes, hyperglycemia). According to the American Diabetes Association, the number of people with diabetes in the United States will increase from 23.7 million to 44.1 million by 2034. Diabetes-related costs account for 12% of the World Health Organization's budget. As the prevalence rate of this disease increases, the need for a new generation of technology to help manage the disease with better portability and greater sensitivity becomes apparent.

Diabetes is a chronic disease in which the clinical symptoms begin in the body, months or even years before the symptoms appear. The reason for this may be that a certain number of cells in each organ and system may maintain the normal function of that organ for a relatively long time (despite the gradual damage and destruction of cells), and this may cause the clinical signs, pathology and biochemistry changes remain hidden from the individual or their doctor for a long time. Since the progression of chronic diseases is often slow and gradual, specific biomarkers may be used to detect the disease in the early stages and before the onset of symptoms.

Screening diabetic patients between the onset of diabetes and the time of clinical detection (Lead Time) is essential to reduce the risk of functional disorders due to diabetes. Conventional glucometers measure the level of blood glucose. However, individuals may have diabetes but have an average level of blood sugar; consequently, utilizing glucometers cannot detect diabetes in its early stages.

Commercially available diagnostic kits that are used in medical laboratories function based on the level and dynamic changes of natural autoantibodies and anti-idiotypic antibodies in the body. The dynamic changes of autoantibodies and self-reactive lymphocytes are called immunculus. In other words, immunculus is the mapping of the body's immunochemical codes. Doctors may utilize immunculus to distinguish a situation in which a person is not yet sick from a condition in which the individual is no longer healthy.

Commercial early detection kits may show a profile of autoantibodies concentration, and concentration of each antibody is not measured. In other words, in commercial early detection kits, the trend of autoantibodies changes over a period of three months or six months is investigated and then the results may be analyzed. The conventional immunculus standard testing methods require special laboratory equipment and expertise and are generally performed in a licensed clinical laboratory requiring high costs in providing equipment and human resources. The need to transfer blood samples to a laboratory, prepare blood samples, and test and analyze the results creates a cumbersome, time-consuming, error-prone, and costly process that may reduce the diagnostic value.

One of the disadvantages of existing detection systems may be detecting the disease based on one parameter. However, in the early detection of diabetes, which is a systemic disease that may affect several organs, it is necessary to examine several biomarkers simultaneously and therefore multiparameter detection tests are required. Furthermore, due to the possibility of human error in the early detection test for diabetes by immunculus kits, the relatively high cost of the kit for screening tests, unbearable, unavailable, time-consuming and multi-step test, the long response time of the diagnostic equipment, sample volume constraints, and the need for skilled labor, there is a need for access to a sensitive, stable, and reproducible system for measuring biomarkers of early detection of diabetes with a shorter response time.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description and the drawings.

According to one or more exemplary embodiments, the present disclosure is directed to an exemplary nano-biosensor system for early detection of diabetes. An exemplary nano-biosensor system may include a plurality of electrochemical immunosensors. Each exemplary electrochemical immunosensor of an exemplary plurality of electrochemical immunosensors may include a plurality of electrodes, where the plurality of electrodes may include a working electrode, a counter electrode, and a reference electrode. In an exemplary embodiment, the counter electrode may include a gold layer and the reference electrode may include a silver layer.

An exemplary working electrode may include a base gold surface, a porous layer of polyaniline conductive polymer that may be deposited onto an exemplary base gold surface, gold nanoparticles that may be deposited inside pores of an exemplary porous layer of polyaniline conductive polymer, L-glutathione reduced linkers that may be attached from respective thiol group ends of exemplary L-glutathione reduced linkers to exemplary gold nanoparticles, and a specific antigen that may be connected to carboxyl group ends of exemplary L-glutathione reduced linkers. An exemplary specific antigen may include at least one of recombinant insulin protein, insulin receptor protein, and recombinant glial fibrillary acidic protein.

An exemplary nano-biosensor system may further include a multiplexed microfluidic system that may be connected in fluid communication with an exemplary plurality of electrochemical immunosensors. An exemplary multiplexed microfluidic system may include a top layer and a multiplexed microfluidic substrate placed below an exemplary top layer. In an exemplary embodiment, an exemplary top layer may include a first layer of at least one of plastic, glass, polymer, and combinations thereof. In an exemplary embodiment, an exemplary top layer may include a sample inlet port and a plurality of electrochemical probe inlets. In an exemplary embodiment, an exemplary sample inlet port may include an opening in an exemplary first layer. In an exemplary embodiment, an exemplary sample inlet port may be used to receive a sample there through. In an exemplary embodiment, an exemplary sample may include at least one of a serum sample and a whole blood sample. In an exemplary embodiment, an exemplary plurality of electrochemical probe inlets may include a plurality of openings in an exemplary first layer. In an exemplary embodiment, each electrochemical probe inlet of an exemplary plurality of electrochemical probe inlets may be used to receive an electrochemical detection probe there through.

In an exemplary embodiment, an exemplary multiplexed microfluidic substrate may include a capillary microfluidic pump connected in fluid communication with an exemplary sample inlet port, a plurality of microfluidic channels connected in fluid communication with an exemplary capillary microfluidic pump, a plurality of electrochemical detection chambers, and a plurality of electrochemical probe storage chambers. An exemplary capillary microfluidic pump may be used to receive an exemplary sample from an exemplary sample inlet port. In an exemplary embodiment, each electrochemical detection chamber of an exemplary plurality of electrochemical detection chambers may be connected in fluid communication with a respective microfluidic channel of an exemplary plurality of microfluidic channels. Each exemplary electrochemical detection chamber of an exemplary plurality of electrochemical detection chambers may encompass a respective plurality of electrodes of a respective electrochemical immunosensor of an exemplary plurality of electrochemical immunosensors. An exemplary capillary microfluidic pump may further be used to pump an exemplary received sample into an exemplary plurality of electrochemical detection chambers through an exemplary plurality of microfluidic channels. In an exemplary embodiment, each electrochemical probe storage chamber of an exemplary plurality of electrochemical probe storage chambers may include one or more cylindrical container receiving an exemplary electrochemical detection probe therein through a respective electrochemical probe inlet of an exemplary plurality of electrochemical probe inlets. In an exemplary embodiment, each electrochemical probe storage chamber may be in in fluid communication with a respective electrochemical detection chamber of an exemplary plurality of electrochemical detection chambers. In an exemplary embodiment, an exemplary electrochemical detection probe may be transferred from an exemplary plurality of electrochemical probe storage chambers into an exemplary respective plurality of electrochemical detection chambers. In an exemplary embodiment, at least a portion of an exemplary sample may be exposed to each plurality of exemplary electrodes that may be disposed within each respective exemplary electrochemical detection chamber of an exemplary plurality of electrochemical detection chambers.

In an exemplary embodiment, each electrochemical immunosensor of an exemplary plurality of electrochemical immunosensors may further include a sensor substrate. In an exemplary embodiment, an exemplary sensor substrate may include a layer of at least one of ceramic, a flexible polymer, and glass. In an exemplary embodiment, an exemplary plurality of electrodes may be disposed on an exemplary sensor substrate.

In an exemplary embodiment, each electrochemical immunosensor of an exemplary plurality of electrochemical immunosensors may further include a plurality of conductive paths formed on an exemplary sensor substrate and an electron transfer cable. In an exemplary embodiment, a first end of each conductive path of an exemplary plurality of conductive paths may be connected to a respective electrode of an exemplary plurality of electrodes. In an exemplary embodiment, an exemplary electron transfer cable may be connected to opposing second ends of an exemplary plurality of conductive paths. In an exemplary embodiment, an exemplary electron transfer cable may be used to connect an exemplary plurality of electrodes to an external measuring device.

In an exemplary embodiment, each electrochemical immunosensor of an exemplary plurality of electrochemical immunosensors may further include a plurality of conductive paths formed on an exemplary sensor substrate and a multiplexed fixed converter. In an exemplary embodiment, a first end of each conductive path of an exemplary plurality of conductive paths may be connected to a respective electrode of an exemplary plurality of electrodes. In an exemplary embodiment, an exemplary multiplexed fixed converter may be connected to opposing second ends of an exemplary plurality of conductive paths. In an exemplary embodiment, an exemplary multiplexed fixed converter may be used to connect an exemplary plurality of electrodes to an external measuring device.

In an exemplary embodiment, an exemplary capillary microfluidic pump may include an input section, a trapezoidal pressure increasing section, and an outlet. In an exemplary embodiment, an exemplary input section may include a first plurality of capillary columns arranged in a square-shaped area. In an exemplary embodiment, an exemplary input section may be connected in fluid communication with an exemplary sample inlet port to receive an exemplary sample. In an exemplary embodiment, an exemplary trapezoidal pressure increasing section may include a second plurality of capillary columns arranged in a trapezoidal area connected to an exemplary input section. In an exemplary embodiment, an exemplary trapezoidal pressure increasing section may receive an exemplary sample from an exemplary input section and increase a pressure of an exemplary received sample. In an exemplary embodiment, an exemplary outlet may include a channel connected in fluid communication between an exemplary trapezoidal pressure increasing section and an exemplary plurality of microfluidic channels. In an exemplary embodiment, an exemplary outlet may be used to distribute an exemplary pumped sample into an exemplary plurality of microfluidic channels.

In an exemplary embodiment, an exemplary nano-biosensor system may further include a separation membrane disposed between an exemplary sample inlet port and an exemplary input section of an exemplary capillary microfluidic pump. In an exemplary embodiment, an exemplary separation membrane may include a biocompatible membrane. In an exemplary embodiment, an exemplary separation membrane may include pores with an average membrane pore size being able to segregate particles with molecular weights ranging from 500 Da to 1000 Da thereon. In an exemplary embodiment, an exemplary separation membrane may be used to separate impurities from an exemplary sample received through an exemplary sample inlet port.

In an exemplary embodiment, an exemplary base gold surface of an exemplary working electrode may include a circular surface with a diameter in a range of 2 to 4 mm. In an exemplary embodiment, an exemplary counter electrode may include a quarter of a ring with an external diameter of 5 to 7 mm and an inner diameter of 3 to 5 mm. In an exemplary embodiment, an exemplary reference electrode may include a quarter of a ring with an external diameter of 5 to 7 mm and an inner diameter of 3 to 5 mm. In an exemplary embodiment, nonspecific sites of an exemplary immobilized specific antigen may be blocked with bovine serum albumin (BSA), where exemplary nonspecific sites may include unreacted sites with an exemplary specific antigen on surface of an exemplary working electrode.

In an exemplary embodiment, each microfluidic channel of an exemplary plurality of microfluidic channels may include a channel with a width of 200 to 600 microns and a height of 20 to 50 microns.

In an exemplary embodiment, an exemplary electrochemical detection probe may include an electrolyte solution. In an exemplary embodiment, an exemplary electrochemical detection probe may include a solution of Potassium hexacyanoferrate (III) (K₃Fe(CN)₆) in phosphate-buffered saline (PBS).

In an exemplary embodiment, an exemplary nano-biosensor system may further include a bottom supporting layer placed below an exemplary multiplexed microfluidic substrate. In an exemplary embodiment, an exemplary bottom supporting layer may include a second layer of at least one of plastic, glass, polymer, and combinations thereof. In an exemplary embodiment, an exemplary bottom supporting layer may include a plurality of recessed slots on one edge of an exemplary second layer. In an exemplary embodiment, each recessed slot of an exemplary plurality of recessed slots may receive and hold a respective electrochemical immunosensor of an exemplary plurality of electrochemical immunosensors. In an exemplary embodiment, an exemplary bottom supporting layer may further include a smooth surface portion that may form a bottom portion of an exemplary plurality of electrochemical detection chambers.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for illustration and description only and are not intended as a definition of the limits of the present disclosure. Embodiments of the present disclosure will now be described by way of example in association with the accompanying drawings in which:

FIG. 1 illustrates a schematic top view of a nano-biosensor system for early detection of diabetes, consistent with one or more exemplary embodiments of the present disclosure;

FIG. 2 illustrates an exploded top view of a nano-biosensor system for early detection of diabetes, consistent with one or more exemplary embodiments of the present disclosure;

FIG. 3 illustrates a schematic top view of an electrochemical immunosensor, consistent with one or more exemplary embodiments of the present disclosure;

FIG. 4 illustrates schematic views of different stages of preparation of a working electrode, consistent with one or more exemplary embodiments of the present disclosure;

FIG. 5 illustrates a schematic top view of a capillary microfluidic pump, consistent with one or more exemplary embodiments of the present disclosure;

FIG. 6A illustrates a field emission electron microscope (FE-SEM) image of an exemplary gold layer, consistent with one or more exemplary embodiments of the present disclosure;

FIG. 6B illustrates an FE-SEM image of an exemplary PANI-modified gold layer, consistent with one or more exemplary embodiments of the present disclosure;

FIG. 6C illustrates an FE-SEM image of an exemplary gold nanoparticle-modified surface, consistent with one or more exemplary embodiments of the present disclosure;

FIG. 6D illustrates an FE-SEM image of an exemplary working electrode surface, consistent with one or more exemplary embodiments of the present disclosure;

FIG. 7A illustrates 2-dimensional (2D) and 3-dimensional (3D) atomic force microscope (AFM) images of an exemplary gold layer, consistent with one or more exemplary embodiments of the present disclosure;

FIG. 7B illustrates 2D and 3D AFM images of an exemplary PANI-modified gold layer, consistent with one or more exemplary embodiments of the present disclosure;

FIG. 7C illustrates 2D and 3D AFM images of an exemplary gold nanoparticle-modified surface, consistent with one or more exemplary embodiments of the present disclosure;

FIG. 7D illustrates 2D and 3D AFM images of an exemplary working electrode surface, consistent with one or more exemplary embodiments of the present disclosure;

FIG. 8 illustrates a Raman spectroscopy spectrum for an exemplary base gold surface, a Raman spectroscopy spectrum for an exemplary PANI-modified gold surface, and a Raman spectroscopy spectrum for an exemplary gold nanoparticle-modified surface, consistent with one or more exemplary embodiments of the present disclosure;

FIG. 9 illustrates a Fourier transform infrared spectroscopy (FTIR) spectrum for an exemplary base gold surface, an FTIR spectrum for an exemplary PANI-modified gold surface, and an FTIR spectrum for an exemplary gold nanoparticle-modified surface, consistent with one or more exemplary embodiments of the present disclosure;

FIG. 10A illustrates a calibration diagram of an exemplary electrochemical immunosensor correlating insulin antibody concentration with an exemplary generated response current obtained by differential pulse voltammetry (DPV) method, consistent with one or more exemplary embodiments of the present disclosure;

FIG. 10B illustrates a calibration diagram of an exemplary electrochemical immunosensor correlating insulin antibody concentration with an exemplary generated response current obtained by square wave voltammetry (SWV) method, consistent with one or more exemplary embodiments of the present disclosure;

FIG. 10C illustrates a calibration diagram of an exemplary electrochemical immunosensor correlating insulin receptor antibody concentration with an exemplary generated response current obtained by DPV method, consistent with one or more exemplary embodiments of the present disclosure;

FIG. 10D illustrates a calibration diagram of an exemplary electrochemical immunosensor correlating insulin receptor antibody concentration with an exemplary generated response current obtained by SWV method, consistent with one or more exemplary embodiments of the present disclosure;

FIG. 10E illustrates a calibration diagram of an exemplary electrochemical immunosensor correlating glial fibrillary acidic protein antibody concentration with an exemplary generated response current obtained by DPV method, consistent with one or more exemplary embodiments of the present disclosure;

FIG. 10F illustrates a calibration diagram of an exemplary electrochemical immunosensor correlating glial fibrillary acidic protein antibody concentration with an exemplary generated response current obtained by SWV method, consistent with one or more exemplary embodiments of the present disclosure;

FIG. 11A illustrates a system selectivity diagram for insulin antibody obtained by DPV method, consistent with one or more exemplary embodiments of the present disclosure;

FIG. 11B illustrates a system selectivity diagram for insulin receptor antibody obtained by DPV method, consistent with one or more exemplary embodiments of the present disclosure; and

FIG. 11C illustrates a system selectivity diagram for glial fibrillary acidic protein antibody obtained by DPV method, consistent with one or more exemplary embodiments of the present disclosure.

### DETAILED DESCRIPTION

The novel features which are believed to be characteristic of the present disclosure, as to its structure, organization, use and method of operation, together with further objectives and advantages thereof, will be better understood from the following discussion.

The present disclosure is directed to exemplary embodiments of a system for early detection of diabetes that may be developed in response to the global necessity and challenge for detection of diabetes in its early stages by an easy and cost-effective method with improved sensitivity and accuracy. An exemplary system may be developed based on simultaneous measuring of several diabetes-specific biomarkers by utilizing a multiplexed nano-biosensor system. An exemplary multiplexed nano-biosensor system may include one or more electrochemical immunosensors with various biorecognition elements and a multiplexed microfluidic mechanism that may be connected in fluid communication with one or more exemplary electrochemical immunosensors. An exemplary multiplexed microfluidic mechanism may be utilized to direct serum or whole blood to one or more exemplary electrochemical immunosensors, where one or more exemplary electrochemical immunosensors may be utilized to measure the concentration of antibodies within the received serum or whole blood.

An exemplary electrochemical immunosensor of one or more exemplary electrochemical immunosensors of an exemplary multiplexed nano-biosensor system may include a modified three-electrode electrochemical immunosensor with specific antigens immobilized on a surface of an exemplary electrochemical immunosensor. An exemplary modified three-electrode electrochemical immunosensor may include a working electrode, a reference electrodes, and a counter electrode. As used herein, immobilizing specific antigens on a surface of an exemplary electrochemical immunosensor may refer to immobilizing specific antigens on a surface of an exemplary working electrode of an exemplary electrochemical immunosensor. Exemplary antigens may include at least one of recombinant insulin protein, insulin receptor protein, and recombinant glial fibrillary acidic protein.

In practice, an exemplary multiplexed microfluidic mechanism may be utilized to receive a biological sample, optionally separate impurities from an exemplary biological sample, and transfer an exemplary biological sample to exemplary electrochemical immunosensors of an exemplary multiplexed nano-biosensor system. Specific antibodies within an exemplary received biological sample related to early detection of diabetes may interact with specific antigens immobilized on surfaces of exemplary electrochemical immunosensors and changes within electrical characteristics of exemplary surfaces of exemplary electrochemical immunosensors may be measured by utilizing external electrical characterization devices connected in signal communication with exemplary electrochemical immunosensors. Here, antibody concentrations related to each specific antigen may be determined based on calibration diagrams. In other words, an exemplary system for early detection of diabetes may allow for assessing the concentration of antibodies in the serum or whole blood to detect diabetes before the appearance of clinical symptoms. Such assessment of concentration of antibodies in a biological sample by immobilizing antigens on a nano-biosensor system may be in contrast with common immunosensors where antibodies are utilized as biorecognition elements to measure the amount of antigen in a fluidic sample.

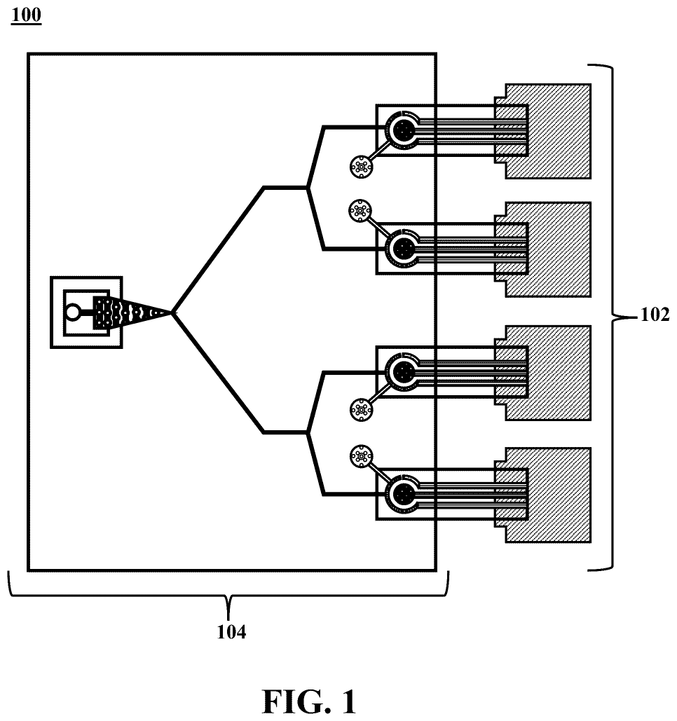

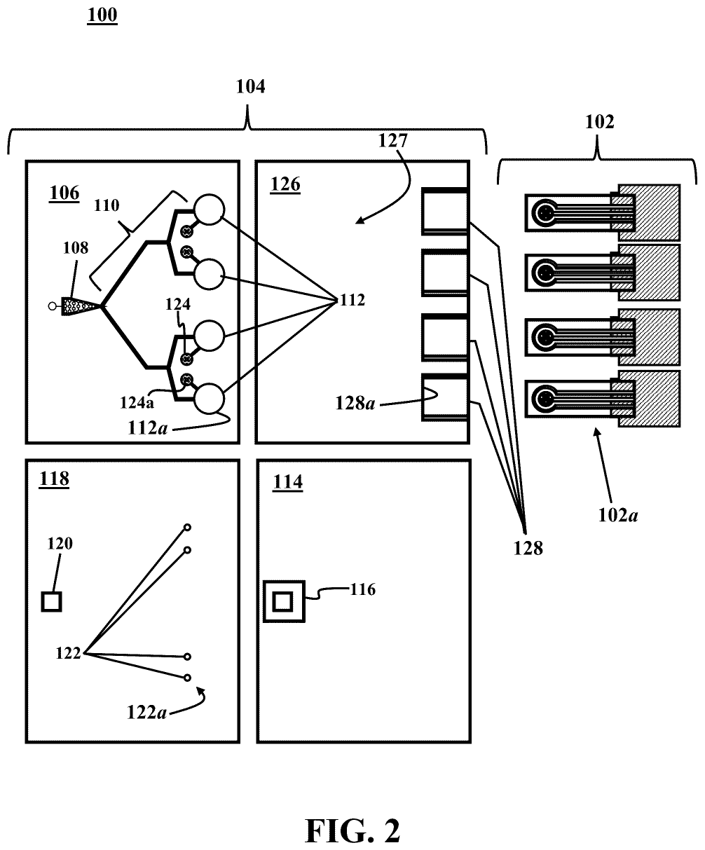

FIG. 1 illustrates a schematic top view of a nano-biosensor system 100 for early detection of diabetes, consistent with one or more exemplary embodiments of the present disclosure. FIG. 2 illustrates an exploded top view of nano-biosensor system 100 for early detection of diabetes, consistent with one or more exemplary embodiments of the present disclosure. In an exemplary embodiment, nano-biosensor system 100 may include a plurality of electrochemical immunosensors 102 with various biorecognition elements and a multiplexed microfluidic system 104 that may be connected in fluid communication with plurality of electrochemical immunosensors 102. In an exemplary embodiment, multiplexed microfluidic system 104 may be utilized to direct a serum or a whole blood sample to plurality of electrochemical immunosensors 102, where plurality of electrochemical immunosensors 102 may be utilized to measure the concentration of antibodies within the received serum or whole blood.

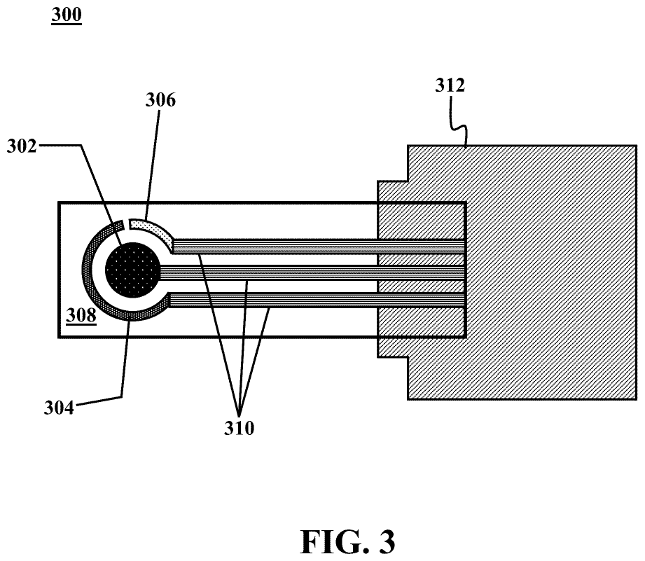

FIG. 3 illustrates a schematic top view of an electrochemical immunosensor 300, consistent with one or more exemplary embodiments of the present disclosure. In an exemplary embodiment, electrochemical immunosensor 300 may be structurally similar to each electrochemical immunosensor of plurality of electrochemical immunosensors 102. In an exemplary embodiment, electrochemical immunosensor 300 may include a working electrode 302, a counter electrode 304, and a reference electrode 306 that may be formed on a sensor substrate 308 made of ceramic, flexible polymer, glass or other similar suitable materials. As used herein, forming electrodes (302, 304, 306) on sensor substrate 308 may refer to forming electrodes (302, 304, 306) on sensor substrate 308 based on a design mask using a layering method that may involve sputtering, depositing, adhering, and patterning on sensor substrate 308. In an exemplary embodiment, working electrode 302 and counter electrode 304 may be made of gold and reference electrode 306 may be made of silver. In an exemplary embodiment, working electrode 302, counter electrode 304, and reference electrode 306 may be connected to an external electron receiver device by utilizing a conductive network 310. In an exemplary embodiment, conductive network 310 may include three conductive paths that may be made of pure copper, pure aluminum, gold or silver, where each respective conductive path of conductive network 310 may be connected to a respective electrode of working electrode 302, counter electrode 304, and reference electrode 306.

In an exemplary embodiment, working electrode 302, counter electrode 304, and reference electrode 306 may be connected to an electron transfer cable 312 by utilizing conductive network 310. In an exemplary embodiment, electron transfer cable 312 may be utilized to connect electrochemical immunosensor 300 to an external measuring device, such as a potentiostat/galvanostat electrochemical instrument (not illustrated for simplicity). In an exemplary embodiment, electron transfer cable 312 may be replaced with a multiplexed fixed converter that may be utilized to connect one or more electrochemical immunosensors similar to electrochemical immunosensor 300 to an external measuring device.

In an exemplary embodiment, working electrode 302 may include a gold surface that may be modified with polyaniline (PANI) and gold nanoparticles, and may further be functionalized with L-glutathione reduced (GSH). In an exemplary embodiment, working electrode 302 may further include specific antigens for early detection of diabetes that may be immobilized on the modified and functionalized gold surface.

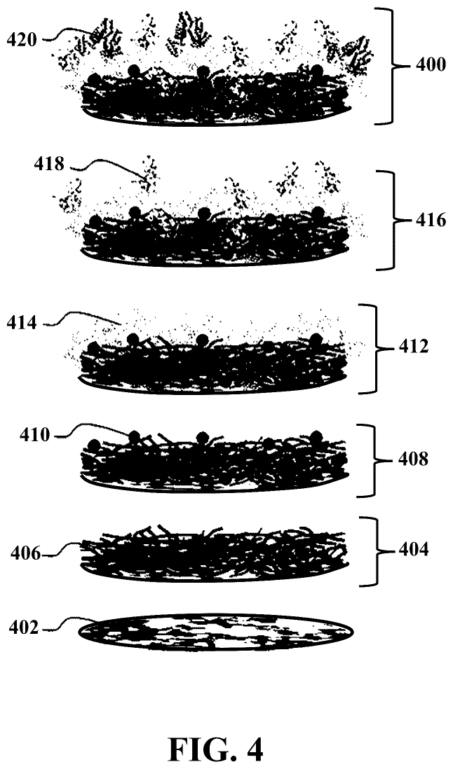

FIG. 4 illustrates schematic views of different stages of preparation of a working electrode 400, consistent with one or more exemplary embodiments of the present disclosure. In an exemplary embodiment, working electrode 400 may be structurally similar to working electrode 302. In an exemplary embodiment, a method for preparing working electrode 400 may include a step of preparing a PANI-modified gold layer 404 by depositing a porous layer of PANI conductive polymer medium onto a base gold surface 402. To this end, in an exemplary embodiment, the step of preparing PANI-modified gold layer 404 may include creating a porous medium of PANI fibers 406 on base gold surface 402 by an electro-polymerization method. In an exemplary embodiment, the electro-polymerization method may involve applying 6 to 10 cyclic voltammograms on base gold surface 402, where voltammograms were applied in 25 mL of a solution consisting of 0.03 M to 0.05 M aniline monomer and 0.5 M to 1 M H₂SO₄. In an exemplary embodiment, PANI electro-polymerization method may be performed at a potential in a range of −0.4 V to +1.2 V, a scan rate of 30 mV/s, and a potential step of 10 mV. The obtained surface or layer may be washed with deionized water and then allowed to dry under room temperature to obtain a PANI-modified gold layer that may be structurally similar to PANI-modified gold layer 404.

In an exemplary embodiment, the method for preparing working electrode 400 may further include a step of obtaining a gold nanoparticle-modified surface 408 by electrically depositing gold nanoparticles 410 inside the pores of PANI-modified gold layer 404. In an exemplary embodiment, electrically depositing gold nanoparticles 410 inside the pores of PANI-modified gold layer 404 may include applying 30 to 35 cyclic voltammograms at the optimum conditions on PANI-modified gold layer 404 in 25 mL solution consisting of 1 mM to 5 mM HAuCl₄, 0.5 M to 1 M H₂SO₄, and 0.1 mM to 0.5 mM NaCl. In an exemplary embodiment, gold nanoparticles may be electrodeposited at a potential range of 100 mV/s and a potential step of 10 mV. In an exemplary embodiment, the obtained layer or surface may be washed with deionized water and may be dried with nitrogen gas to obtain a gold nanoparticle-modified surface that may be structurally similar to gold nanoparticle-modified surface 408.

In an exemplary embodiment, the method for preparing working electrode 400 may further include a step of obtaining a GSH-functionalized surface 412 by functionalizing gold nanoparticle-modified surface 408 with GSH linkers 414. Here, an exemplary GSH linker may include a thiol group end for attaching to the surface of gold nanoparticles 410 and a carboxyl group end for binding to an antigen. In an exemplary embodiment, functionalizing gold nanoparticle-modified surface 408 with GSH linkers 414 may involve dropping 5 μL of a solution of GSH on gold nanoparticle-modified surface 408. GSH includes thiol groups and carboxylic acid groups with high affinity with gold nanoparticles and protein. As mentioned before, the thiol group of GSH interacts with gold through formation of Au—S bonds. In addition, the carboxylic acid groups of GSH provide the attachment to amine groups of protein.

In an exemplary embodiment, the method for preparing working electrode 400 may further include a step of obtaining an antigen-immobilized surface 416 by immobilizing specific antigens 418 for early detection of diabetes on GSH-functionalized surface 412 by covalent bonding. In an exemplary embodiment, immobilizing specific antigens 418 on GSH-functionalized surface 412 may include exposing GSH-functionalized surface 412 to a mixture of antigen phosphate buffer solution and N-hydroxy succinimide (NHS), followed by an incubation for 120 min at 37° C. In an exemplary embodiment, NHS may function as a catalyst and may facilitate the interaction of the carboxylic acid groups of the monolayer of GSH to amine groups of immunomolecules. In an exemplary embodiment, the mixture of antigen and NHS may be prepared by mixing 200 μl of an antigen solution in PBS and 300 μl of an NHS solution by utilizing a vortex mixer in a 5 ml tube for 10 seconds. Then, the mixture was incubated for 60 min at room temperature.

In an exemplary embodiment, following immobilization of specific antigens 418 on GSH-functionalized surface 412, antigen-immobilized surface 416 may be washed with 3 mL of a mixture of PBS and polysorbate 20 for 3 minutes by utilizing a vortex mixer. Then, antigen-immobilized surface 416 was further washed with 3 mL of pure PBS for 3 minutes to ensure the removal of non-adsorbed proteins off of antigen-immobilized surface 416. In an exemplary embodiment, washing antigen-immobilized surface 416 may be followed by drying antigen-immobilized surface 416 by utilizing nitrogen gas.

In an exemplary embodiment, the method for preparing working electrode 400 may further include a step of blocking nonspecific sites of antigen-immobilized surface 416 by utilizing bovine serum albumin (BSA) 420 to obtain working electrode 400. In an exemplary embodiment, the step of blocking nonspecific sites of antigen-immobilized surface 416 may include blocking the unreacted active functional groups from previous steps by casting 5 μl of a BSA solution on antigen-immobilized surface 416 and allowing BSA to react with non-specific bonds for 45 min at 37° C. at water saturation condition. In an exemplary embodiment, prepared working electrode 400 may then be rinsed for 3 min with a mixture of PBS and Polysorbate 20 and washed again with PBS for 3 min to eliminate non-specific binding. In an exemplary embodiment, working electrode 400 may then be dried with nitrogen gas, and then the prepared working electrode 400 may optionally be refrigerated at 4° C. until use.

In an exemplary embodiment, responsive to the exposure of the prepared working electrode 400 to a serum or whole blood sample, specific antibodies related to early detection of diabetes within the sample may interact with specific antigens 418. In exemplary embodiments, such interaction between specific antibodies and specific antigens 418 may cause a change in electrical activity and electrical resistance of working electrode 400, which may be measured by utilizing an exemplary electrical characterization device.

In an exemplary embodiment, multiplexed microfluidic system 104 may include a multiplexed microfluidic substrate 106 that may be designed and used to direct a sample to plurality of electrochemical immunosensors 102, where each electrochemical immunosensor of electrochemical immunosensors 102 may be structurally similar to electrochemical immunosensor 300 with a working electrode that may be structurally similar to working electrode 400. In an exemplary embodiment, multiplexed microfluidic substrate 106 may include a capillary microfluidic pump 108 that may be used to receive an exemplary serum or whole blood sample and pump the received exemplary serum or whole blood sample through a plurality of microfluidic channels 110 into a corresponding plurality of electrochemical detection chambers 112. In an exemplary embodiment, capillary microfluidic pump 108 may be used to pump a serum or whole blood sample by utilizing capillary force. In an exemplary embodiment, the pumped sample may be exposed to a respective electrochemical immunosensor of plurality of electrochemical immunosensors 102 that may be disposed within each corresponding electrochemical detection chamber of plurality of electrochemical detection chambers 112. In an exemplary embodiment, each detection chamber 112a of plurality of electrochemical detection chambers 112 may include an edged hollow compartment encompassing an exemplary plurality of electrodes of an electrochemical immunosensor 102a of plurality of electrochemical immunosensors 102.

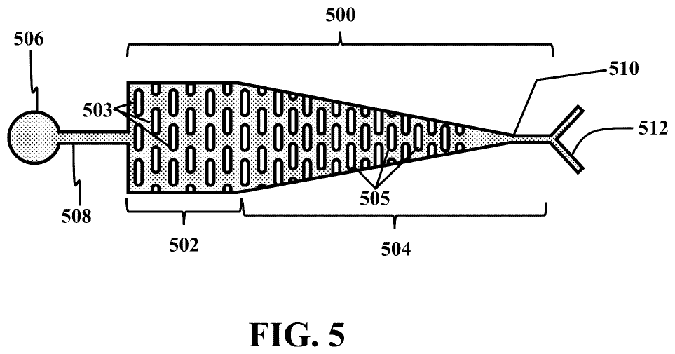

FIG. 5 illustrates a schematic top view of a capillary microfluidic pump 500, consistent with one or more exemplary embodiments of the present disclosure. In an exemplary embodiment capillary microfluidic pump 500 may be structurally similar to capillary microfluidic pump 108. In an exemplary embodiment, capillary microfluidic pump 500 may include an input section 502 and a pressure increasing section 504. In an exemplary embodiment, input section 502 may include a first plurality of capillary columns 503 arranged in a square-shaped area of capillary microfluidic pump 500. In an exemplary embodiment, capillary microfluidic pump 500 may be connected to an intake 506 by utilizing a short microfluidic channel 508. In an exemplary embodiment, intake 506 may be connected to input section 502 and may be used to clean capillary microfluidic pump 500 or vent the trapped air within capillary microfluidic pump 500. In an exemplary embodiment, a serum or whole blood sample may be received within capillary microfluidic pump 500 from a top side of input section 502 of capillary microfluidic pump 500 and the pressure of the received sample may be increased within pressure increasing section 504. In an exemplary embodiment, pressure increasing section 504 may have a trapezoidal shape to prevent formation of static points along capillary microfluidic pump 500. In an exemplary embodiment, pressure increasing section 504 may include a second plurality of capillary columns 505 arranged in a trapezoidal area of capillary microfluidic pump 500. In an exemplary embodiment, pressurized sample may be discharged from an outlet 510 of capillary microfluidic pump 500. In an exemplary embodiment, a three-way connection 512 may be connected to outlet 510 to distribute the pressurized sample into plurality of microfluidic channels 110. In an exemplary embodiment, microfluidic channels 110 may include symmetrical tree-shaped channels that may be used to distribute an exemplary pumped sample simultaneously and equally to electrochemical detection chambers 112.

In an exemplary embodiment, multiplexed microfluidic system 104 may further include a separation layer 114 that may be disposed on top of multiplexed microfluidic substrate 106. In an exemplary embodiment, separation layer 114 may include a separation membrane 116 that may be located at an inlet of capillary microfluidic pump 108. In an exemplary embodiment, separation membrane 116 may separate impurities from an exemplary serum or whole blood sample. In an exemplary embodiment, separation membrane 116 may have an average membrane pore size; allowing for segregating particles with molecular weights ranging from 500 to 1000 Da. In an exemplary embodiment, separation membrane 116 may be a membrane made of cellulose ester.

In an exemplary embodiment, multiplexed microfluidic system 104 may further include a top layer 118 that may be disposed on top of separation layer 114. In an exemplary embodiment, top layer 118 may include a first layer of at least one of plastic, glass, polymer, and combinations thereof. In an exemplary embodiment, top layer 118 may include a sample inlet port 120 that may include an opening in top layer 118. In an exemplary embodiment, sample inlet port 120 may be positioned on top of separation membrane 116 and the inlet of capillary microfluidic pump 108. Such positioning of sample inlet port 120 my allow for an exemplary sample to be introduced into multiplexed microfluidic system 104 via sample inlet port 120 and then pass through separation membrane 116 into capillary microfluidic pump 108.

In an exemplary embodiment, top layer 118 may further include electrochemical probe inlets 122 that may allow for entering exemplary electrochemical detection probes into corresponding electrochemical probe storage chambers 124 formed within multiplexed microfluidic substrate 106. In an exemplary embodiment, electrochemical probe inlets 122 may include a plurality of openings in top layer 118. In an exemplary embodiment, an exemplary electrochemical detection probe may include an electrolyte solution. In an exemplary embodiment, an exemplary electrochemical detection probe may include a solution of Potassium hexacyanoferrate (III) (K₃Fe(CN)₆) in PBS.

In an exemplary embodiment, each electrochemical probe storage chamber 124 of electrochemical probe storage chambers 124 may include one or more cylindrical containers embedded in multiplexed microfluidic substrate 106 receiving an exemplary electrochemical detection probe therein through a corresponding electrochemical probe inlet 122a of plurality of electrochemical probe inlets 122. In an exemplary embodiment, an exemplary electrochemical detection probe may be transferred from plurality of electrochemical probe storage chambers 124 into corresponding plurality of electrochemical detection chambers 112.

In an exemplary embodiment, top layer 118 may seal a top side of multiplexed microfluidic substrate 106. To this end, top layer 118 may be attached to multiplexed microfluidic substrate 106 by utilizing a two-stage oxygen plasma approach. In an exemplary embodiment, top layer 118 may be made of a suitable material such as plastic, glass, or preferably a polymer by a method such as molding, laser formation, or three-dimensional printing. In an exemplary embodiment, the two-stage oxygen plasma approach may include washing multiplexed microfluidic substrate 106 with a 70 v/v % ethanol solution to clean any contamination and then subjecting multiplexed microfluidic substrate 106 and top layer 118 to oxygen plasma treatment at 0.6 mbar vacuum for 30 seconds. In an exemplary embodiment, the two-stage oxygen plasma approach may further include placing multiplexed microfluidic substrate 106 and top layer 118 in a 30 v/v % aqueous solution of acrylic acid at room temperature to create COOH functional groups. In an exemplary embodiment, the two-stage oxygen plasma approach may further include drying multiplexed microfluidic substrate 106 and top layer 118 at 40° C. for 5 minutes. In an exemplary embodiment, the two-stage oxygen plasma approach may further include subjecting multiplexed microfluidic substrate 106 and top layer 118 to oxygen plasma treatment at 0.6 mbar vacuum for another 3 minutes. Then, multiplexed microfluidic substrate 106, separation layer 114, and top layer 118 may be aligned with and bound to each other.

In an exemplary embodiment, multiplexed microfluidic system 104 may further include a bottom supporting layer 126 that may be positioned below multiplexed microfluidic substrate 106 and may support plurality of electrochemical immunosensors 102 and form a bottom portion of electrochemical detection chambers 112. In an exemplary embodiment, bottom supporting layer 126 may include a solid and strong layer that may be made of a rigid materials such as plastic, glass, or a rigid polymer. As used herein, bottom supporting layer 126 as a support for plurality of electrochemical immunosensors 102 may refer to bottom supporting layer 126 including a plurality of recessed slots 128 that may be formed at one edge of bottom supporting layer 126. In an exemplary embodiment, each recessed slot of plurality of recessed slots 128 may be adapted to receive and hold a corresponding electrochemical immunosensor of plurality of electrochemical immunosensors 102. For example, a recessed slot 128a of plurality of recessed slots 128 may be shaped and sized such that electrochemical immunosensor 102a may be removably placed within recessed slot 128a. In an exemplary embodiment, to allow for easy placement and removal of each corresponding electrochemical immunosensor of plurality of electrochemical immunosensors 102 in and out of each respective recessed slot of plurality of recessed slots 128, width of each respective recessed slot of plurality of recessed slots 128 may be slightly (500-1000 micrometers) larger than the width of a corresponding electrochemical immunosensor of plurality of electrochemical immunosensors 102. In an exemplary embodiment, bottom supporting layer 126 may include a smooth surface portion 127 forming an exemplary bottom portion of electrochemical detection chambers 112.

In an exemplary embodiment, nano-biosensor system 100 may be utilized as a device for simultaneous detection of different biomarkers of human biological fluids. In an exemplary embodiment, such simultaneous detection of different biomarkers may be possible by immobilizing a respective antigen on each respective electrochemical immunosensor of plurality of electrochemical immunosensors 102. This way, each respective electrochemical immunosensor of plurality of electrochemical immunosensors 102 may be utilized for detection of a specific antibody. In an exemplary embodiment, nano-biosensor system 100 may be utilized to perform an electrochemical detection method. In an exemplary embodiment, such electrochemical method performed by utilizing nano-biosensor system 100 may benefit from ease of signal quantification. As mentioned before, response current generated in electrochemical immunosensors 102 of nano-biosensor system 100 when exposed to a sample may be correlated to a desired biomarker concentration within the sample. For example, the response current generated in an electrochemical immunosensor of electrochemical immunosensors 102 may be linearly correlated with the logarithm of a specific biomarker concentration. Consequently, by obtaining calibration data of pure antibodies in the detection range, the biomarker concentration in an unknown sample may be obtained by utilizing a calibration diagram.

### Example

In this example, a multiplexed nano-biosensor system that may be structurally similar to nano-biosensor system 100 was fabricated and then utilized for early detection of diabetes. An exemplary electrochemical immunosensor that was structurally similar to electrochemical immunosensor 300 was prepared. The exemplary electrochemical immunosensor included a working electrode structurally similar to working electrode 302, a counter electrode structurally similar to counter electrode 304, a reference electrode structurally similar to reference electrode 306, and a three-electrode conductive network structurally similar to conductive network 310. In this example, a substrate was obtained by sputtering a layer of gold on a glass plate. The exemplary substrate was then coated completely with a photoresist and was patterned using UV radiation on a mask designed for the exemplary gold layer. As a result, the locations of the working electrode and the counter electrode were revealed on the substrate. The gold layer on other glass areas were etched using a wet etching method, and the remaining photoresist was removed with acetone. Then, a layer of silver was sputtered on the substrate, and a secondary photoresist was spin coated on the substrate. After that, photolithography was performed by UV radiation on a designed mask for the silver layer, and the reference electrode area and the three-electrode conductive network were revealed, and finally, the silver layer on other regions was removed.

After the electrochemical immunosensor substrate was prepared as described in the previous paragraph, surface modification steps of the working electrode of the electrochemical immunosensor were performed. First, a surface of the gold layer of the working electrode which may be similar to base gold surface 402 was cleaned by a cyclic voltammetry method in the potential range of −0.4 to +1.4 volts with the scan rate of 100 mV·s¹ and the potential step of 2.94 mV in sulfuric acid solution with the concentration of 50 to 100 mM in 6 to 10 cycles. Then, the gold layer was washed with deionized water and dried at room temperature.

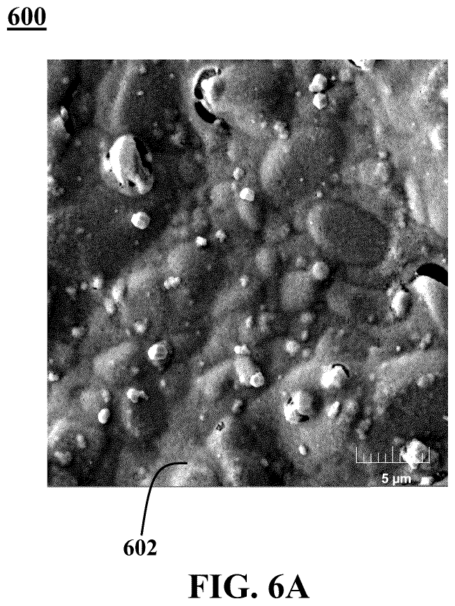

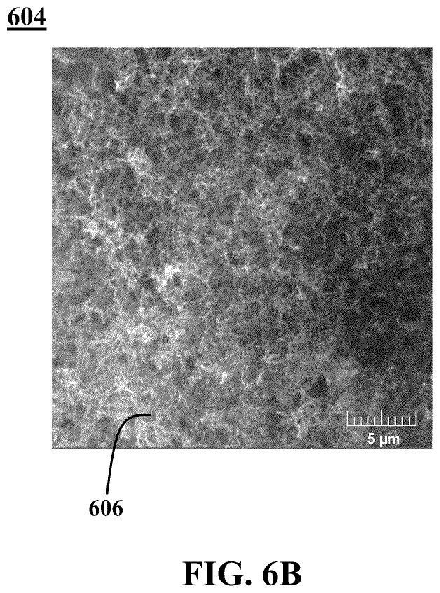

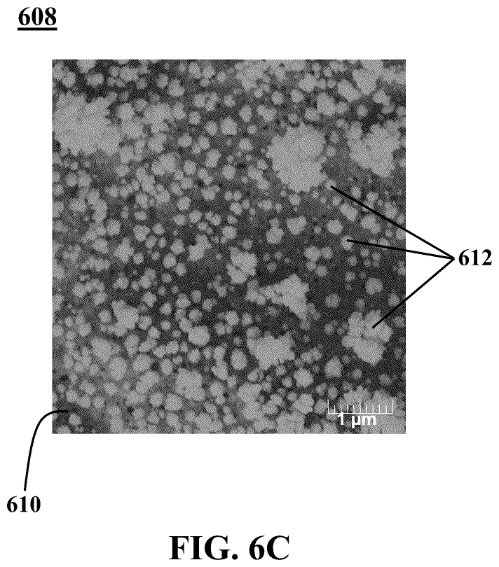

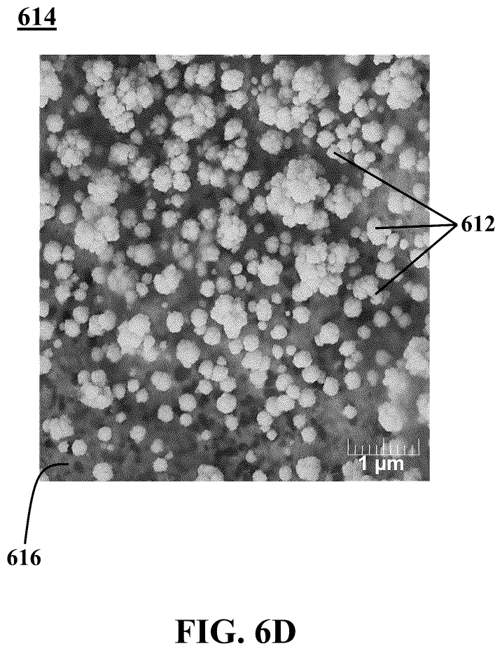

FIG. 6A illustrates a field emission electron microscope (FE-SEM) image 600 of exemplary gold layer 602, consistent with one or more exemplary embodiments of the present disclosure. A morphology of a biosensing platform may play an essential role in the stability, sensitivity, and reproducibility properties of an exemplary nano-biosensor. It is evident in FIG. 6A that gold layer 602 does not have a flat and uniform surface morphology and instead gold layer 602 includes some holes on its surface that could decrease the reproducibility of an exemplary biosensor.

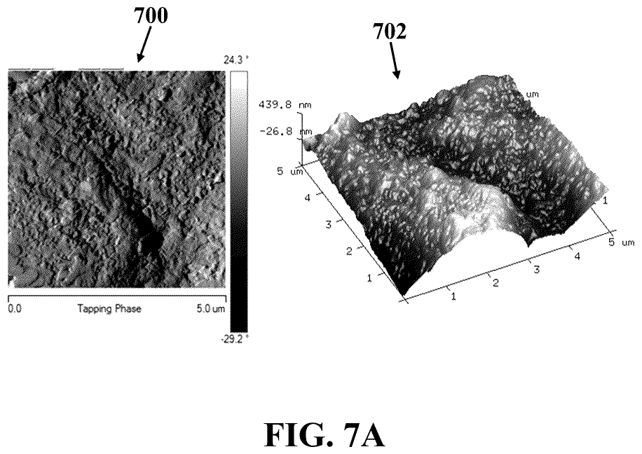

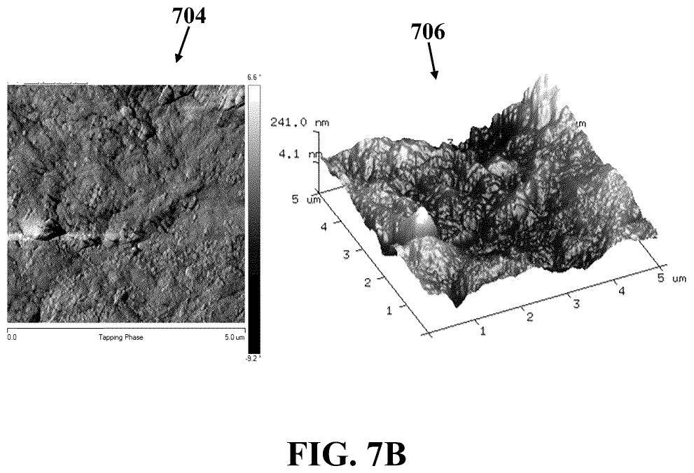

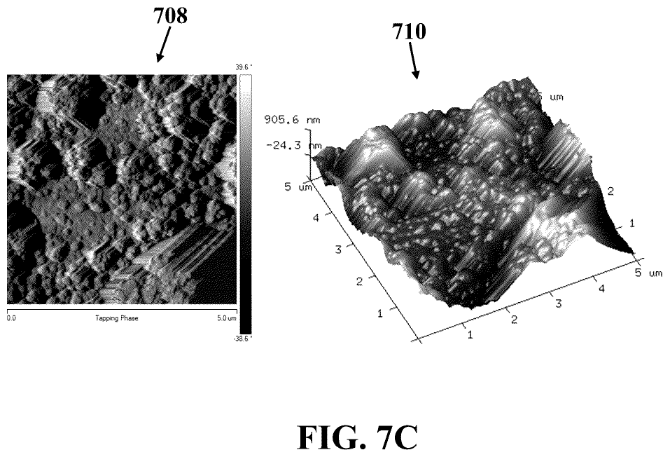

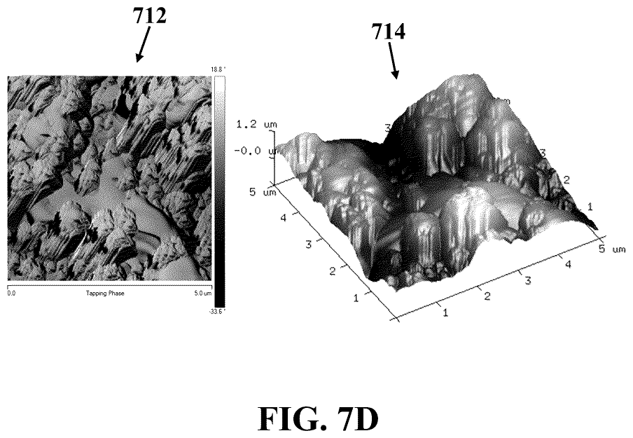

FIG. 7A illustrates 2-dimensional (2D) and 3-dimensional (3D) atomic force microscope (AFM) images 700 and 702 of gold layer 602, consistent with one or more exemplary embodiments of the present disclosure. Referring to FIG. 7A, the brightest regions outline the peak points of the electrode surface, and the dark districts represent the valleys or nanocomposite pores.

After cleaning surface of base gold layer 602, polyaniline was electro polymerized by a cyclic voltammetry method in the potential range of −0.4 to +1.2 volts with the scan rate of 30 mV·s¹ and the potential step of 10 mV in a solution of 0.03 to 0.05 M aniline monomer and 0.5 to 1 M sulfuric acid in 6 to 10 cycles to create a porous medium of polyaniline fibers on gold layer 602 surface and obtain a PANI-modified gold layer 606 (illustrated in FIG. 6B) similar to PANI-modified gold layer 404. PANI is a conductive polymer with excellent environmental stability, significant electroactivity, uncommon doping/dedoping chemistry, controllable chemical and electrical properties, long shelf life, reversible redox activity, easy and diverse synthesis techniques, and low costs. PANI has fast electron transport dynamics and has good electrochemical activity. Due to extremely effective surface area and small penetration depth for target analytes, nanostructured PANI exhibits excellent sensitivity and short response time in biosensor applications. PANI may also serve as a good matrix for immobilizing bio-components on an exemplary electrode surface.

FIG. 6B illustrates an FE-SEM image 604 of exemplary PANI-modified gold layer 606, consistent with one or more exemplary embodiments of the present disclosure. Referring to FIG. 6B, PANI-modified gold layer 606 includes a porous nanofiber medium is visible, which was made by electro-polymerization of polyaniline. FIG. 7B illustrates 2D and 3D AFM images 704 and 706 of PANI-modified gold layer 606, consistent with one or more exemplary embodiments of the present disclosure. FIG. 7B indicates that PANI-modified gold layer 606 has a uniform grain size and even surface topography than bare gold layer 602. By comparison of AFM images of two steps shown in FIGS. 7A-7B, it can be concluded that the grain size of an exemplary electrode including PANI-modified gold layer 606 is smaller than that of a gold electrode (GE) including gold layer 602, which may indicate that an exemplary PANI/GE has a more uniform grain size, smoother and more regular surface morphology in comparison with an exemplary GE.

After obtaining PANI-modified gold layer 606, gold nanoparticles 612 (illustrated in FIG. 6C) were electrodeposited by a cyclic voltammetry method in a potential range between −0.5 to +1.5 volts with the scan rate of 100 mV·s¹ and the potential step of 10 mV in a solution of 1 to 5 mM of gold salt, 0.5 to 1 molar of sulfuric acid and 0.1 to 0.5 mM of sodium chloride for 30 to 35 cycles. Gold nanoparticles 612 were deposited in the pore of the porous medium of polyaniline and on surface of polyaniline fibers of PANI-modified gold layer 606 to obtain a gold nanoparticle-modified surface 610 (illustrated in FIG. 6C) similar to gold nanoparticle-modified surface 408. An exemplary obtained surface was then washed with de-ionized water and dried with a stream of nitrogen gas.

FIG. 6C illustrates an FE-SEM image 608 of exemplary gold nanoparticle-modified surface 610, consistent with one or more exemplary embodiments of the present disclosure. FIG. 6C demonstrates that polyaniline nanofibers of PANI-modified gold layer 606 are decorated with gold nanoparticles 612. The morphology of nano-biosensor for immobilization of antigen is subordinate to dispersion of Au nanoparticles 612 in an exemplary polymer matrix of PANI-modified gold layer 606. The excellent and uniform dispersant for Au nanoparticles 612 on an exemplary modified electrode could increase sensitivity of an exemplary nano-biosensor dramatically. The particle size of Au nanoparticles 612 is almost 50-100 nm. Gold nanoparticles 612 on polyaniline nanofiber of PANI-modified gold layer 606 give an expansive surface zone for the immobilization of antigen thereon.

FIG. 7C illustrates 2D and 3D AFM images 708 and 710 of gold nanoparticle-modified surface 610, consistent with one or more exemplary embodiments of the present disclosure. The nanoporous granular rough morphology of gold nanoparticle modified electrode reveals that gold nanoparticles 612 are uniformly decorated an exemplary polyaniline surface of PANI-modified gold layer 606. By increasing the electrodeposition cycles, the shape of growing gold nanoparticles 612 changes to tree-shaped and the sharper valley in the topography can be seen in FIG. 7C.

After obtaining gold nanoparticle-modified surface 610, the surface was functionalized by utilizing a L-glutathione reduced (GSH) linker. Here, GSH linker was used to bind the antigen to an exemplary modified substrate. A solution of 10 to 20 mM GSH in phosphate buffered saline solution was prepared. Then, 5 to 10 μl of the solution was poured on gold nanoparticle-modified surface 610 to obtain a GSH functionalized surface similar to GSH-functionalized surface 412. Then, the GSH-functionalized surface was placed in an incubator at 37° C. saturated with water vapor for 1 hour to dry the electrochemical immunosensor substrate.

A 100 to 150 mM NHS solution in PBS buffer was initially produced for the immobilization stage of the antigen as the receptor on the GSH-functionalized surface. Then, 300 to 600 μl of NHS solution was combined with 200 to 400 μl of specific antigen solution for early detection of diabetes at a concentration of 10 to 20 μg·ml⁻¹ in a microtube and mixed with a vortex mixer for 10 seconds. The microtube was stored at room temperature for 1 to 2 hours. The GSH-functionalized surface was then placed in the microtube and incubated at 37° C. for 2.5 to 5 hours to obtain an antigen-immobilized surface similar to antigen-immobilized surface 416. To get rid of unbound NHS materials, a PBS buffer solution containing Tween-20 with a volume percentage of 0.05 to 0.1 was prepared, and the antigen-immobilized surface was washed with this solution for 3 minutes by utilizing a vortex mixer. Then, the antigen-immobilized surface was washed with PBS buffer 0.01 to 0.05 M at a pH of about 7.4 for 3 minutes using a vortex mixer, and then the antigen-immobilized surface was dried using a stream of nitrogen gas.

In the next step, to block the nonspecific active sites of the functionalized surface, a 5 μl of BSA solution with the concentration of 0.2 to 0.5 percent in PBS buffer was poured on the antigen-immobilized surface and was incubated at 37° C. for 45 minutes in water vapor saturation to obtain the working electrode. A washing procedure was performed on the working electrode and repeated in two steps with PBS buffer solution containing polysorbate-20 and sterile PBS solution. The working electrode was prepared to perform clinical tests with serum or whole blood and calibration tests with specific antibodies related to the early detection of diabetes. The working electrode was dried with a stream of nitrogen and kept in a refrigerator at 4° C. until usage.

FIG. 6D illustrates an FE-SEM image 614 of an exemplary working electrode surface 616, consistent with one or more exemplary embodiments of the present disclosure. FIG. 6D demonstrates the relatively uniform distribution of gold nanoparticles 612 on an exemplary porous polyaniline medium of working electrode surface 616. Besides, cloudy parts on working electrode surface 616 might be related to an attachment of biomolecules to gold nanoparticles 612.

FIG. 7D illustrates 2D and 3D AFM images 712 and 714 of working electrode surface 616, consistent with one or more exemplary embodiments of the present disclosure. As evident in FIG. 7D, after the immobilization of insulin antigen and precursor biological material for the preparation of an exemplary nano-biosensor, the rough picture of the modified electrode changes into another normal topography containing many larger island structures, revealing the immobilization of antigen and also confirming BSA attachment to block non-binding sites on an exemplary nano-biosensor. The change in nano-biosensor roughness compared to gold nanoparticle-modified surface 610 also confirms immobilization of the antigen and BSA attachment on working electrode surface 616, to good extent.

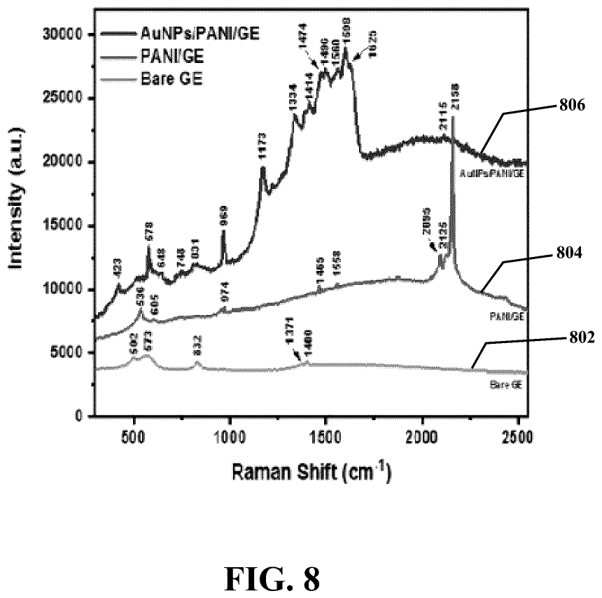

FIG. 8 illustrates a Raman spectroscopy spectrum 802 for an exemplary base gold surface of gold layer 602, a Raman spectroscopy spectrum 804 for an exemplary PANI-modified gold surface of PANI-modified gold layer 606, and a Raman spectroscopy spectrum 806 for exemplary gold nanoparticle-modified surface 610, consistent with one or more exemplary embodiments of the present disclosure. Here, Raman measurements were examined for different preparation steps to find some more information on the biosensor platform's molecular structure. The spectral profile of AuNPs/PANI/GE contains predominant bands, ascribed to conductive emeraldine salt and gold nanoparticles. They are positioned as follows: 1598 cm⁻¹ (C—C stretching vibrations of B units), 1560 cm⁻¹ (C═C stretching vibration in Q rings), 1496 cm⁻¹ (N—H bending vibration), 1334 cm⁻¹ (C—N+• vibration of delocalized polaronic structure), 1223 cm⁻¹ (C—N stretching vibrations of B-units), 1173 cm⁻¹ (C—H bending in-plane vibrations of Q segments), 831 cm⁻¹ (deformation vibration of Q rings), 748 cm⁻¹ (C—C out-of-plane deformation vibrations of PANI polaronic form) and 578 cm⁻¹ (in-plane deformation vibrations of amine groups of bipolaronic PANI structure). Besides these bands, the relatively strong bands at 1414 cm⁻¹ and 578 cm⁻¹, a shoulder at ˜1625 cm⁻¹, assigned to substituted phenazine-like segments, is also observed. When the spectral profiles of AuNPs/PANI/GE nanocomposite are compared to spectral profiles of PANI/GE nanocomposite, it can be seen that there are signs for modestly high content of phenazine-like segments in nanocomposites or their interaction with the surface of AuNPs; thus, Raman scattering is regionally surface-enhanced. This enhancement leads to better detection of phenazine-like segments.

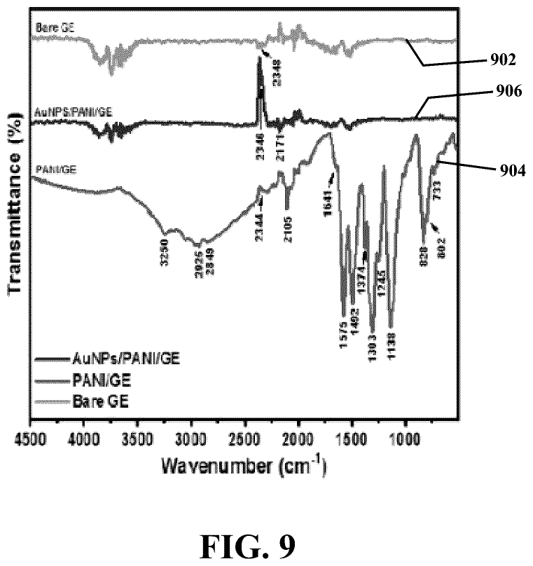

FIG. 9 illustrates a Fourier transform infrared spectroscopy (FTIR) spectrum 902 for an exemplary base gold surface of gold layer 602, an FTIR spectrum 904 for an exemplary PANI-modified gold surface of PANI-modified gold layer 606, and an FTIR spectrum 906 for exemplary gold nanoparticle-modified surface 610, consistent with one or more exemplary embodiments of the present disclosure. FTIR-ATR is a mature and compelling technique for elemental analysis and functional group identification. The FTIR spectrum of bare gold electrode did not show any significant vibrational stretches reported in the literature. Therefore, it was used as the background subtraction for subsequent measurements. As can be depicted, the band characteristic for PANI/GE is positioned as follows: in the high wavenumber region at 3250 cm⁻¹ corresponding to N—H stretching, at 2925 cm⁻¹ (aromatic C—H stretching vibration), at 1575 cm⁻¹ relating to C═C stretching vibration in the quinonoid (Q) polaronic structures -Q-NH+•—, at about 1492 cm-1 assigning to C═C stretching vibration of benzenoid (B) ring in —NH—B—NH— units), at 1303 cm⁻¹ corresponding to C—N+• stretching in Q segments, at about 1245 cm⁻¹ relating to C—N stretching vibrations of secondary aromatic amine, and at about 828 cm⁻¹ (C—H out-of-plane deformation vibration of 1,4-disubstituted benzene ring). Likewise, the band observed for the PANI/GE at 1138 cm⁻¹ is attributed to the stretching vibration in charged polymer units —B—NH+=Q- and(or) B—NH+•—B, which is related to the electrical conductivity of PANI and a high degree of electron delocalization in PANI chains.

The infrared spectrum of modified AuNps/PANI/GE appeared similar to the clean gold surface spectrum because of gold nanoparticles electrodeposition on the polyaniline nanofiber. The change of bands in the range of 2100 cm⁻¹ to 2400 cm⁻¹ shows electronic interaction between conductive polyaniline and gold nanoparticles, which is manifested by a drastic modification of the plasmon absorption band of gold nanoparticles along with a significant enhancement in the emission characteristics. Also, FIG. 9 indicates a little shift in the PANI characteristic bands at the 2105 cm⁻¹ and 2344 cm⁻¹ with the Au nanoparticles electrodeposition, suggesting the presence of interaction between the PANI functional groups and the nanoparticles. Moreover, the addition of gold nanoparticles will increase the biosensor performance due to the ability of the antigen functional group to interact with the gold nanoparticle surface. This strategy could improve electrode conductivity and surface area.

After the exemplary electrochemical immunosensor, which was structurally similar to electrochemical immunosensor 300 was prepared as described in the preceding paragraphs, the electrochemical immunosensor was subjected to calibration tests. Here, for performing the calibration tests, various antibody solutions were prepared with different concentrations of 0, 0.1, 0.5, 1, 5, 10, 50, 100 units per ml of PBS solution and then 5 to 10 μl of each antibody solution with a specified concentration was poured on the prepared electrochemical immunosensor and was evaluated after 1 to 2 hours. This process was repeated for all the above mentioned concentrations. In this example, the electrochemical immunosensor evaluation for obtaining the calibration diagram was performed by two approaches of differential pulse voltammetry (DPV) and square wave voltammetry (SWV). DPV method was conducted in a solution containing the electrochemical probe with the modulation of 0.025 volts, modulation time of 0.05 seconds, the potential step of 0.005 volts, the voltage range of +0.5 to +1.5 volts, and the scan rate of 50 mV. The SWV method was performed in a solution containing an electrochemical probe with a modulation amplitude of 0.02 V, a potential step of 0.005 V, a frequency of 25 Hz, and a voltage range of −1.2 to +1.0 V.

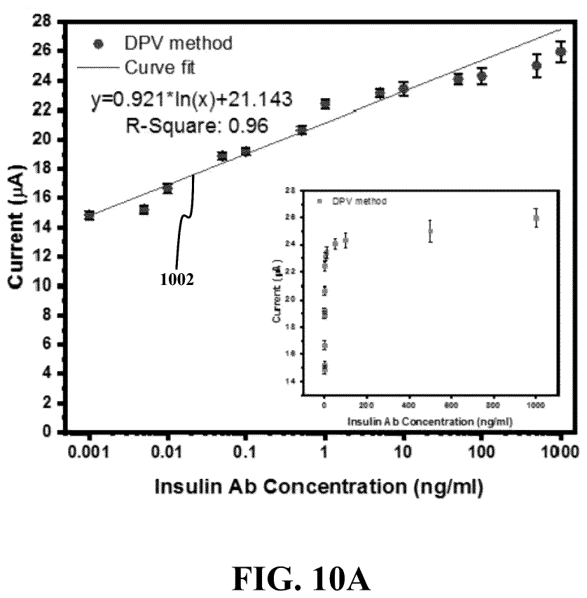

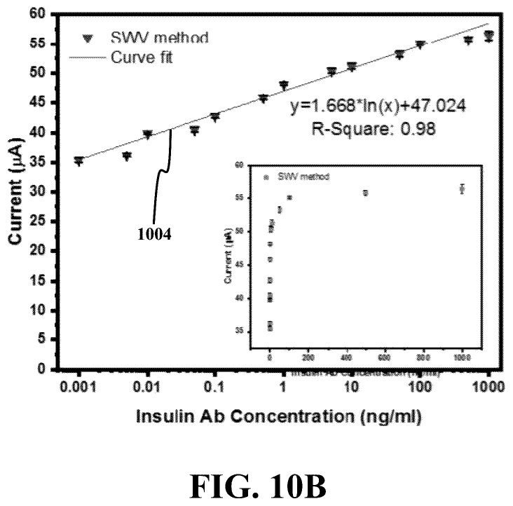

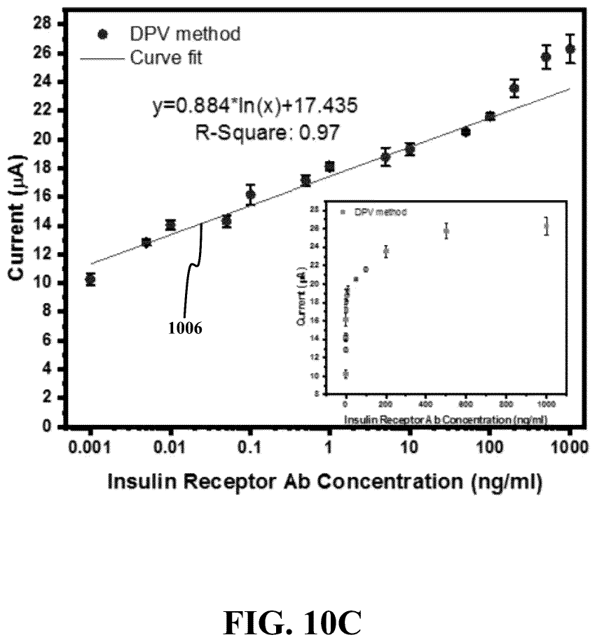

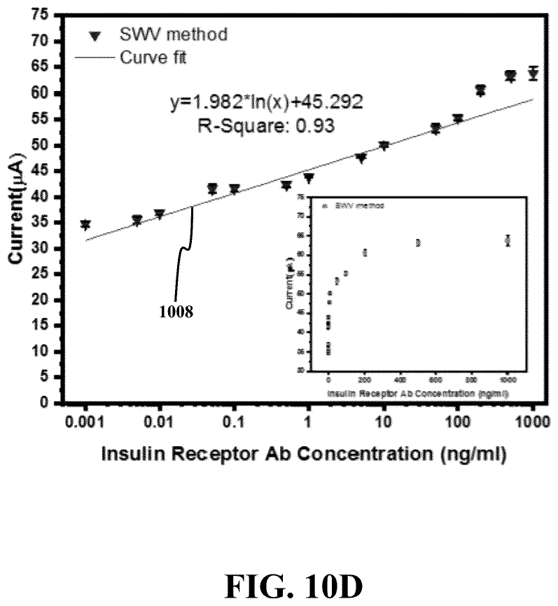

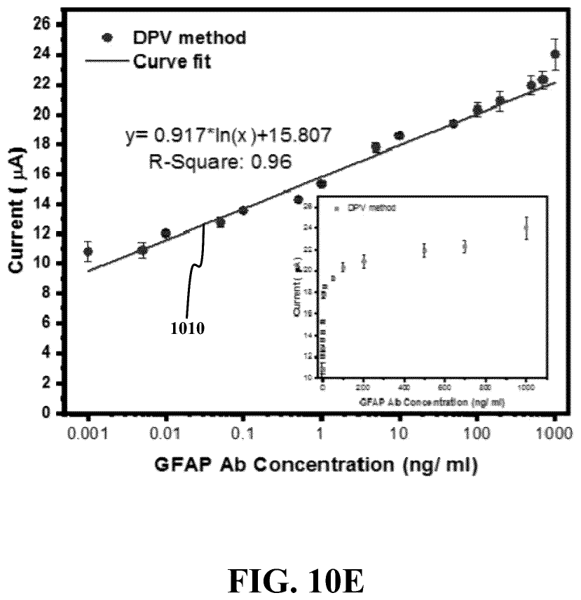

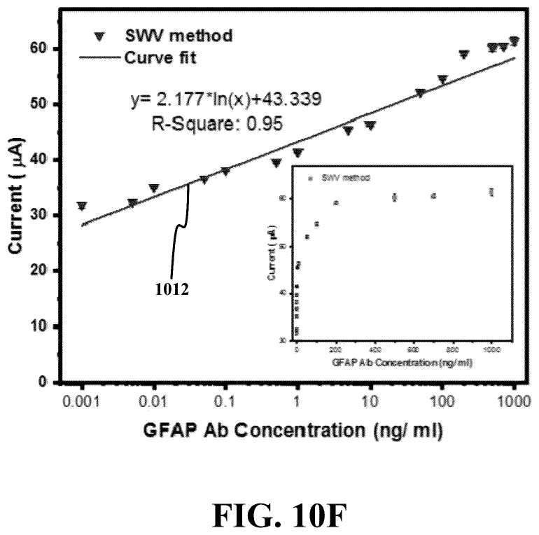

FIG. 10A illustrates a calibration diagram 1002 of the electrochemical immunosensor correlating insulin antibody concentration with the generated response current obtained by DPV method, consistent with one or more exemplary embodiments of the present disclosure. FIG. 10B illustrates a calibration diagram 1004 of the electrochemical immunosensor correlating insulin antibody concentration with the generated response current obtained by SWV method, consistent with one or more exemplary embodiments of the present disclosure.

FIG. 10C illustrates a calibration diagram 1006 of the electrochemical immunosensor correlating insulin receptor antibody concentration with the generated response current obtained by DPV method, consistent with one or more exemplary embodiments of the present disclosure. FIG. 10D illustrates a calibration diagram 1008 of the electrochemical immunosensor correlating insulin receptor antibody concentration with the generated response current obtained by SWV method, consistent with one or more exemplary embodiments of the present disclosure.

FIG. 10E illustrates a calibration diagram 1010 of the electrochemical immunosensor correlating glial fibrillary acidic protein antibody concentration with the generated response current obtained by DPV method, consistent with one or more exemplary embodiments of the present disclosure. FIG. 10F illustrates a calibration diagram 1012 of the electrochemical immunosensor correlating glial fibrillary acidic protein antibody concentration with the generated response current obtained by SWV method, consistent with one or more exemplary embodiments of the present disclosure.

Referring to FIGS. 10A-10F, the results of the electrochemical response of the electrochemical immunosensor for different concentrations of insulin antibody, insulin receptor antibody, and glial fibrillary acidic protein antibody in the same laboratory conditions illustrate that the response current increases with enhancing antibody concentration. These results are due to forming an immune complex between antibodies and antigens and the change in surface catalytic activity that improves the electron transfer at the boundary between the electrochemical immunosensor and the electrochemical detection probe. As a result, the response current of the electrochemical immunosensor is linearly related to the logarithm of the antibody concentration.

The sensitivity of the electrochemical insulin immunosensor using the DPV method is 7.368 μA·ml·ng⁻¹·cm⁻², and its detection limit is 10.29 ng·ml⁻¹. The sensitivity of the electrochemical insulin immunosensor using the SWV method is 13.344 μA·ml·ng⁻¹·cm⁻², and its limit of detection is 1.21 ng·ml⁻¹. The sensitivity of the electrochemical insulin receptor immunosensor using the DPV method is 7.072 μA·ml·ng⁻¹·cm⁻², and its detection limit is 0.128 ng·ml⁻¹. The sensitivity of the electrochemical insulin receptor immunosensor using the SWV method is 15.856 μA·ml·ng⁻¹·cm⁻², and its detection limit is 1.036 ng·ml⁻¹. The sensitivity of electrochemical GFAP immunosensor using DPV method is 7.336 μA·ml·ng⁻¹·cm⁻² and its detection limit is 0.012 ng·ml⁻¹. The sensitivity of the electrochemical GFAP immunosensor using the SWV method is 17.416 μA·ml·ng⁻¹·cm⁻², and its detection limit is 1.271 ng·ml⁻¹.

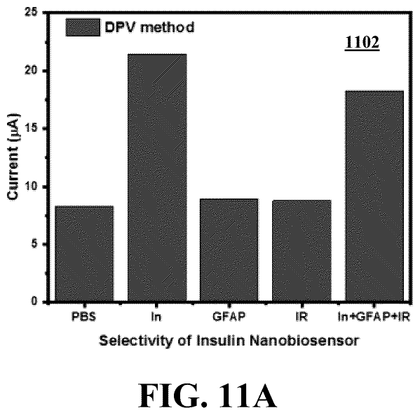

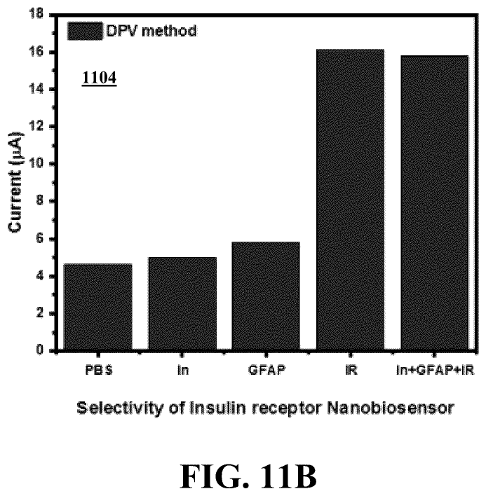

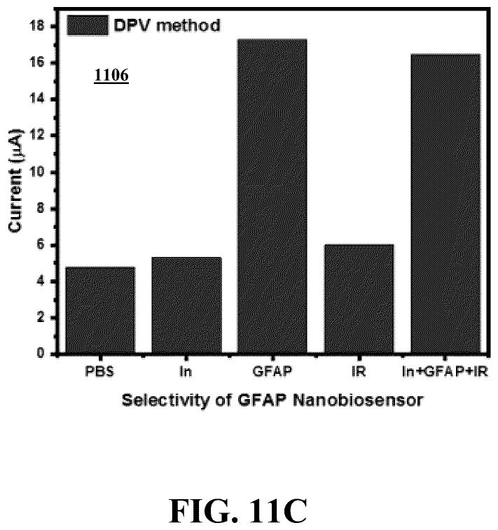

FIG. 11A illustrates a system selectivity diagram 1102 for insulin antibody obtained by DPV method, consistent with one or more exemplary embodiments of the present disclosure. FIG. 11B illustrates a system selectivity diagram 1104 for insulin receptor antibody obtained by DPV method, consistent with one or more exemplary embodiments of the present disclosure. FIG. 11C illustrates a system selectivity diagram 1106 for glial fibrillary acidic protein antibody obtained by DPV method, consistent with one or more exemplary embodiments of the present disclosure.

Referring to FIGS. 11A-11C, selectivity of the insulin immunosensor relative to insulin is significant, and a minimal decrease is observed with the combination of other antibodies, which indicates reasonable specificity of insulin antibody with immobilized insulin antigen. The insulin receptor immunosensor was also selective against the other two antibodies (insulin and glial fibrillary acidic protein) and does not have an interference reaction with them. The glial fibrillary acidic protein antibody immunosensor is selective against the other two antibodies (insulin and insulin receptor).

The next exemplary step in preparation procedure of an exemplary nano-biosensor system similar to nano-biosystem 100 was to fabricate a multiplexed microfluidic layer that was structurally similar to multiplexed microfluidic substrate 106. The structure of the multiplexed microfluidic layer of the present example included a capillary microfluidic pump structurally similar to capillary microfluidic pump 500, symmetrical channels in the form of a tree structure similar to that of microfluidic channels 110 with a direct inlet to direct serum or whole blood to at least four electrochemical detection chambers similar to electrochemical detection chambers 112 and four electrochemical probe storage chambers similar to probe storage chambers 124. Here, each microfluidic channel of the multiplexed microfluidic layer may have a width of 200 to 500 μm and a height of 20 to 50 μm, which miniaturized the multiplexed nano-biosensor system and reduced the consumption of serum or whole blood samples. The electrochemical detection chamber may have a 7.5 to 10 mm diameter, and a height that was equal to the thickness of the multiplexed microfluidic layer, which was 3 to 5 mm.

In this example, the multiplexed microfluidic layer was prepared utilizing an exemplary photolithographic method. First, a 3-inch silicon wafer was cleaned with acetone and de-ionized water, then an amount of SU-8 photoresist was poured over the central section of the wafer. The wafer was then rotated on a spin coating system for 30 seconds at a 2000 to 3000 rpm speed to create a photoresist thickness of 20 μm to 50 μm on the surface, then baked gently overnight at 45° C. The designed photomask was then placed on a wafer coated with SU-8 and irradiated with UV light at the intensity of 220 mJ·cm⁻². Next, the secondary curing step was performed at 90° C. immediately after exposure to UV light. When the pattern was clearly visible, the wafer was submerged in Su-8 developer until the non-exposed sections of the photoresist were removed.

In a next exemplary step, the wafer was washed with isopropanol and de-ionized water to remove residual waste. When the multiplexed microfluidic mold was formed on the wafer, the PDMS mixture containing the curing agent and the base silicone elastomer in a ratio of 1:9 was poured on the prepared mold and then two hours of degassing process and also two hours of baking process at 75° C. were carried out. Then, the multiplexed microfluidic layer prepared from PDMS was separated from the relevant mold, and the mold pattern was shown on the PDMS layer accurately, and finally, using the oxygen plasma device, in two steps, the packing process of the multiplexed microfluidic layer with another layer was conducted.

The multiplexed nano-biosensor system disclosed in this example further included a lower supporting layer structurally similar to bottom supporting layer 126, an upper layer structurally similar to top layer 118, and an impurity separation membrane structurally similar to separation membrane 116 formed on separation layer 114. Here, the lower supporting layer had a thickness of 3 mm and had at least four electrochemical immunosensor locations structurally similar to plurality of recessed slots 128 on its edge and was preferably made of polylactic acid. The dimensions of lower supporting layer were 80 to 100 mm in length, 60 to 90 mm in width, and 3.5 to 5 mm in thickness.

The upper layer was made of polylactic acid and had a serum or whole blood inlet in a square shape with the side of 5 to 8 mm and four inputs of electrochemical detection probe with a diameter of 1.5 to 3 mm. Its dimensions are 80 to 100 mm by 60 to 90 mm, and its thickness was 3 to 5 mm, respectively. The membrane for the separation of serum or whole blood impurities was preferably made of cellulose ester with dimensions of 9.75 mm by 10 mm, had a molecular weight cut-off of 500 to 1000 Daltons, and was biocompatible. The impurity separation membrane was attached to the inlet of the capillary pump of the multiplexed microfluidic layer.

In order to investigate the stability of the multiplexed nano-biosensor system, electrochemical immunosensors prepared for all three types of antibodies were functionally tested for two months at different time intervals. The reduction in performance of all three types of antibodies in total is less than 15%, which is desirable. This phenomenon is due to the slight degradation of the immobilized antigens in the electrochemical immunosensor substrate over time.

According to one or more exemplary embodiments, an exemplary multiplexed nano-biosensor system for early detection of diabetes may allow for simultaneous detection of low concentrations of several antibodies in a small volume of a biofluidic sample. As used herein, a small volume of a biofluidic sample may refer to 100 to 200 microliters of a biofluidic sample. An exemplary multiplexed nano-biosensor system may be used for mapping immune codes related to the early detection of diabetes based on electrochemical analysis.

The exemplary embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic, e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

What is claimed is:

1. A nano-biosensor system for early detection of diabetes, the nano-biosensor system comprising:
a plurality of electrochemical immunosensors, each respective electrochemical immunosensor comprising a plurality of electrodes, the plurality of electrodes comprising:
a working electrode, comprising:
a base gold surface;
a porous layer of polyaniline conductive polymer deposited onto the base gold surface;
gold nanoparticles deposited inside pores of the porous layer of polyaniline conductive polymer;
L-glutathione reduced linkers attached from respective thiol group ends of the L-glutathione reduced linkers to the gold nanoparticles; and
a specific antigen connected to carboxyl group ends of the L-glutathione reduced linkers;
a counter electrode, the counter electrode comprising gold; and
a reference electrode, the reference electrode comprising silver; and
a multiplexed microfluidic system connected in fluid communication with the plurality of electrochemical immunosensors, the multiplexed microfluidic system comprising:
a top layer comprising a first layer of at least one of plastic, glass, polymer, and combinations thereof, the top layer comprising:
a sample inlet port comprising an opening in the first layer, the sample inlet port configured to receive a sample comprising at least one of a serum sample and a whole blood sample there through;
a multiplexed microfluidic substrate placed below the top layer, comprising:
a capillary microfluidic pump connected in fluid communication with the sample inlet port, the capillary microfluidic pump configured to receive the sample from the sample inlet port;
a plurality of microfluidic channels connected in fluid communication with the capillary microfluidic pump;
a plurality of electrochemical detection chambers, each respective electrochemical detection chamber connected in fluid communication with a respective microfluidic channel of the plurality of microfluidic channels, each respective electrochemical detection chamber comprising an edged hollow compartment encompassing a respective plurality of electrodes of a respective electrochemical immunosensor of the plurality of electrochemical immunosensors, the capillary microfluidic pump further configured to pump the received sample into the plurality of electrochemical detection chambers through the plurality of microfluidic channels; and
a plurality of electrochemical probe storage chambers, each respective electrochemical probe storage chamber comprising one or more cylindrical containers receiving the electrochemical detection probe therein through a respective electrochemical probe inlet of the plurality of electrochemical probe inlets, each respective electrochemical probe storage chamber being in in fluid communication with a respective electrochemical detection chamber of the plurality of electrochemical detection chambers, the electrochemical detection probe being transferred from the plurality of electrochemical probe storage chambers into the respective plurality of electrochemical detection chambers,
wherein, at least a portion of the sample is exposed to each plurality of electrodes disposed within each respective electrochemical detection chamber of the plurality of electrochemical detection chambers.

2. The nano-biosensor system of claim 1, wherein the specific antigen comprises at least one of recombinant insulin protein, insulin receptor protein, and recombinant glial fibrillary acidic protein.

3. The nano-biosensor system of claim 1, wherein each electrochemical immunosensor of the plurality of electrochemical immunosensors further comprises a sensor substrate comprising a layer of at least one of ceramic, a flexible polymer, and glass, the plurality of electrodes disposed on the sensor substrate.

4. The nano-biosensor system of claim 3, wherein each electrochemical immunosensor of the plurality of electrochemical immunosensors further comprises:
a plurality of conductive paths formed on the sensor substrate, a first end of each respective conductive path of the plurality of conductive paths being connected to a respective electrode of the plurality of electrodes; and
an electron transfer cable connected to opposing second ends of the plurality of conductive paths, the electron transfer cable configured to connect the plurality of electrodes to an external measuring device.

5. The nano-biosensor system of claim 3, wherein each electrochemical immunosensor of the plurality of electrochemical immunosensors further comprises:
a plurality of conductive paths formed on the sensor substrate, a first end of each respective conductive path of the plurality of conductive paths being connected to a respective electrode of the plurality of electrodes; and
a multiplexed fixed converter connected to opposing second ends of the plurality of conductive paths, the multiplexed fixed converter configured to connect the plurality of electrodes to an external measuring device.

6. The nano-biosensor system of claim 1, wherein the capillary microfluidic pump comprises:
an input section comprising a first plurality of capillary columns arranged in a square-shaped area, the input section being connected in fluid communication with the sample inlet port to receive the sample;
a trapezoidal pressure increasing section comprising a second plurality of capillary columns arranged in a trapezoidal area connected to the input section, the trapezoidal pressure increasing section receiving the sample from the input section and increase a pressure of the received sample; and
an outlet comprising a channel connected in fluid communication between the trapezoidal pressure increasing section and the plurality of microfluidic channels, the outlet configured to distribute the pumped sample into the plurality of microfluidic channels.

7. The nano-biosensor system of claim 6, further comprising a separation membrane disposed between the sample inlet port and the input section of the capillary microfluidic pump, the separation membrane comprising a biocompatible membrane comprising pores with an average membrane pore size being able to segregate particles with molecular weights ranging from 500 Da to 1000 Da thereon, the separation membrane configured to separate impurities from the sample received through the sample inlet port.

8. The nano-biosensor system of claim 1, wherein the base gold surface comprises a circular surface with a diameter in a range of 2 to 4 mm.

9. The nano-biosensor system of claim 8, wherein the counter electrode comprises a quarter of a ring with an external diameter of 5 to 7 mm and an inner diameter of 3 to 5 mm.

10. The nano-biosensor system of claim 9, wherein the reference electrode comprises a quarter of a ring with an external diameter of 5 to 7 mm and an inner diameter of 3 to 5 mm.

11. The nano-biosensor system of claim 1, wherein nonspecific sites of the immobilized specific antigen are blocked with bovine serum albumin (BSA), the nonspecific sites comprising unreacted sites with the specific antigen on surface of the working electrode.

12. The nano-biosensor system of claim 1, wherein each microfluidic channel of the plurality of microfluidic channels comprises a channel with a width of 200 to 600 microns and a height of 20 to 50 microns.

13. The nano-biosensor system of claim 1, wherein the electrochemical detection probe comprises an electrolyte solution.

14. The nano-biosensor system of claim 13, wherein the electrolyte solution comprises a solution of Potassium hexacyanoferrate (III) (K₃Fe(CN)₆) in phosphate-buffered saline (PBS).

15. The nano-biosensor system of claim 1, further comprising a bottom supporting layer placed below the multiplexed microfluidic substrate, the bottom supporting layer comprising a second layer of at least one of plastic, glass, polymer, and combinations thereof, the bottom supporting layer comprising:
a plurality of recessed slots on one edge of the second layer, each respective recessed slot receiving and holding a respective electrochemical immunosensor of the plurality of electrochemical immunosensors; and
a smooth surface portion of the second layer defining a bottom portion of the plurality of electrochemical detection chambers.
